(19) United States
(12) Patent Application Publication
(10) Pub. No.: US 2023/0000714 A1
(43) Pub. Date: Jan. 5, 2023

(54) APPARATUS AND METHOD FOR PULSE CYCLE PRESSURE MODULATION AND NEGATIVE PRESSURE THERAPY

(71) Applicant: Syncardon LLC, Broomall, PA (US)

(72) Inventor: Vikas Khurana, Broomall, PA (US)

(21) Appl. No.: 17/366,579

(22) Filed: Jul. 2, 2021

(51) Int. Cl. A61H 9/00; A61M 1/00

(52) U.S. Cl. CPC A61H 9/0007; A61M 1/985; A61H 9/0092; A61H 9/0057; A61H 2201/5007; A61H 2230/201; A61H 2201/1238

(57) ABSTRACT

The present invention relates to a system and method for pulse cycle pressure modulation and negative pressure therapy in a cardio synchronous manner to improve distal limb blood flow applying negative or variable pressure to a portion of the body. The invention relates to methods and apparatus for the application of a sequential and gradient pulse wave for treating wounds that are difficult to heal or livelihood limiting claudication and/or ischemic rest pain. An integral part of the present invention includes methods to determine blood flow and effects of treatments on tissue.

### FIELD OF THE INVENTION

The present invention generally relates to an apparatus for pulse cycle pressure modulation and negative pressure therapy in a cardio synchronous manner to improve distal limb blood flow; wherein said apparatus is provisioned for applying a combination of positive pressure to a portion of the body and negative or variable pressure to a portion of the body. Said invention particularly relates to methods and apparatus for the application of a sequential and gradient pulse wave for treating wounds that are difficult to heal or livelihood limiting claudication and/or ischemic rest pain. Said invention also applies to treat microvascular diseases in various segments of the body, like diabetic neuropathy, restless leg syndrome, Berger's disease and Sickle cell disease among others conditions that are difficult to treat. An integral part of the present invention includes methods to determine blood flow and effects of treatments on tissue.

### BACKGROUND OF THE INVENTION

Impaired circulation, i.e., decreased blood supply to tissue, is a common condition that can arise from several causes. For example, atherosclerosis of arteries as well as diabetic small vessel disease can cause ischemic tissue, leading to pain and/or non-healing wounds, a serious condition that can lead to gangrene and/or amputation or functional limb loss. There are other disorders that can have devastating outcomes and can benefit from enhancing the circulation but are not classically related to named vessel-arterial obstruction. These include diabetic and other types of peripheral neuropathy, reflex sympathetic dystrophy, frostbite, chilblains and trauma induced wounds such as those incurred during combat.

It is also well accepted, per the Starling-Landis theory, that, in addition to patent arteries another factor helps facilitate blood flow through capillaries and back to the heart, a low pressure venous system at the distal end of capillaries. That low pressure helps facilitate blood delivery through tissues by providing a low resistance flow system.

Patients with atherosclerosis and other forms of peripheral arterial disease not infrequently have impaired cardiac function. Diastolic heart failure is well known, having as its basis the presence of poorly relaxing myocardium that raises diastolic pressure. That diastolic pressure elevation creates elevated central venous pressure that impedes return of venous blood. Furthermore, impaired venous return may also impede lymphatic flow, since that flow depends on a normally functioning venous system to facilitate lymphatic drainage through the thoracic duct. Impaired lymphatic drainage leads to an excessive build-up of lymphatic fluid in the interstitium, further aggravating tissue hypoxia.

Over the last 20 plus years application of subatmospheric pressure, commonly called negative pressure therapy, has been shown to enhance wound healing. It is well known to treat impaired circulation with pressure devices that squeeze the limb, typically by means of an inflatable pressure cuff or similar device wrapped around the limb. The pressure device moves fluid within the treated limb by applying pressure to only a part of the limb. In the prior art some types of devices are designed to move arterial blood flow downstream. In some prior art the devices move fluid from tissues from distal portions of the limb to proximal portions, eventually to the trunk of the body, to reduce tissue pressure in the limb so as to allow improved blood inflow. These pressure devices thus perform external, non-invasive compression therapy.

The prior art uses at least three techniques to apply the pressure, known as waveforms. The first type of waveform is non-sequential and non-gradient, synchronized with the cardiac cycle, and thus pulsatile. The limb compressing component of the device encloses the leg, but not the foot. This art is illustrated by U.S. Pat. No. 2,690,174, by Maurice Fuchs. An expandable bladder is placed around the limb and inflated to a certain pressure for squeezing the limb and forcing blood to flow distally (at least supposedly) to the end of the limb. This waveform is simple and basic and suffers from several drawbacks. First, the design can use high pressures that thus risks discomfort since the device has no specified method to prevent elevated pressure and/or sustained rather than intermittent squeezing. Second, the entire volume of the bladder, a single compartment, is inflated at the same time, and thus has the potential to push fluid both distally and proximally, rather than only to enhance arterial flow distally to diminish ischemia. This design thus has the potential to undermine the very goal of circulation improvement by pushing fluid toward the trunk.

The second type of waveform is also non-sequential and non-gradient, synchronized with the cardiac cycle and thus pulsatile. Its limb squeezing component, however, does cover the foot. This art is illustrated by U.S. Pat. No. 3,961,625, by Richard S. Dillon. An inflatable plastic enclosure is placed around the entire limb, including the foot, and inflated to a certain pressure for squeezing the limb and forcing blood to flow distally (at least supposedly) to the end of the limb. Like the first type of waveform described above, it is simple and basic, but also suffers from several drawbacks. Because, like the first type of pump design, it squeezes each limb using a single compartment, its drawbacks stem from that design. In addition, the entire limb, including the foot is encased in the plastic enclosure. Thus, the foot is squeezed along with the rest of the limb, impeding blood flow into the very part of the limb most vulnerable to ischemia, the end, at the foot. This design therefore inherently puts the foot at risk for more ischemia. The enclosure covers the entire limb including the foot. It is therefore not available for inspection to assure lack of adverse consequences of squeezing, such as swelling or cyanosis. It is also thus not available to place a blood flow monitoring device or sensor, to determine if indeed squeezing is improving blood flow or to guide adjustment of timing to optimize compression timing. In addition, this design uses a rubber cuff at the proximal end of the limb enclosure to provide a seal. The seal, if too firm can entrap fluid in the leg, leading to undesired swelling. If the seal is too loose, squeezing the limb via a single compartment can allow fluid to exit the limb proximally that is, opposite to the goal of enhancing distal flow, to the ischemic end of the limb.

The third type of waveform is prolonged relative to the duration of the cardiac cycle and of modest strength. This art is illustrated by U.S. Pat. No. 5,218,954, issued to Paul S. van Bemmelen and marketed as the ArtAssist® as well as other devices described below. These include the ArterialFlow System® by AirCast® and the FM-220® device by FlowMedic®. This design is purported to improve circulation by reducing tissue fluid, thereby allowing for more arterial inflow. This design has not been shown to actually lead to important clinical outcomes such as predictable and efficient limb salvage. Furthermore, the form of this design that utilizes foot compression suffers from the same drawbacks as the second type of waveform cited above that squeezes the foot.

The non-sequential waveform device, synchronized with the cardiac cycle, is used in U.S. Pat. No. 4,077,402 to Benjamin et al. A single inflatable compartment is used to squeeze the limb and generate a waveform. The device of said U.S. Pat. No. 4,077,402 and its method of operation involve several drawbacks and do not lead to effective pressure therapy of limb tissue, particularly a wound or edema. Drawbacks of U.S. Pat. No. 4,077,402 include: (i) application of electrographic apparatus and its sensors on the chest of the patient, which can be a substantial practical problem for the health service provider in treating female patients in an outpatient setting; (ii) mounting of such EKG sensors on the chest of patient, can create substantial psychological disturbance to the female patient and it may lead to an abnormal cardiological responses therein; (iii) does not provide compression from proximal region to distal region and thus risks pushing blood retrograde, away from the limb's distal ischemic area; (iv) the negative pressure, which is achieved within the pressure channel of the device and is transferred to the target region of the limb, particularly because it has to be achieved in reference to the atmospheric pressure; and (v) practically can be difficult to interpret the inputs from EKG signals correctly for selecting the best moment to trigger the compression means and also to decide the duration of such compressions.

A non-sequential waveform device, synchronized with the cardiac cycle, is used in U.S. Pat. No. 3,961,625, issued Jun. 8, 1976 to Richard S. Dillon. The device depends on an external source of compressed fluid, as a separate device supplied by the user, to facilitate cuff inflation. The entire limb including the foot is encased in a hard-outer plastic shell, in which is placed a single inflatable enclosure. Access to the foot to monitor blood flow and inspect skin status is thus denied.

Another non-sequential waveform device, also synchronized with the cardiac cycle, is used in U.S. Pat. No. 6,921,373, issued Jul. 26, 2005 to Myron Z. Bernstein. It is described as a peripheral circulation enhancement system. It times the compression cycle to start at or just after the P wave, to push fluids out into venous structures and back to the heart. With the next heart beat the extremity is now void of some of the fluid, presumably forcing from the heart fresh oxygenated blood, thereby promoting circulation through the heart.

Two patents provide non-cardiac cycle compressions that last seconds. U.S. Pat. No. 5,218,954 to Paul S. van Bemmelen (ACI Medical, Inc.) and marketed as the ArtAssist® uses inflatable cells that first compress the foot and ankle, and, a second later compresses the calf. This action is described as almost completely emptying the veins of the foot, ankle and calf. This action is supposed to facilitate easier flow of arterial blood to the toes and blood-deprived tissues by increasing the arterial-venous pressure gradient. Calf compression starts 1 second after foot and ankle compression, thus providing sequential compression, from the distal to proximal aspect of the limb, in the opposite direction to arterial flow. However, because a cuff covers and compresses the foot, blood flow into this most ischemic part of the limb at the end of the arterial tree during compression, which lasts for 4 seconds, is limited. Likewise lack of access to the foot prevents use of inspection and blood flow monitoring to document benefit with treatments. Published reports of benefit range from 30 to 60% and may require several hundred hours of treatments.

Another device, U.S. Pat. No. 6,463,934 to Glenn W. Johnson, Jr. et al (ArterialFlow System by AirCast®, now part of DJO Global of Vista, Calif.) also describes delivery of rapid inflation, graduated sequential compression from the foot to the calf, in reverse direction to arterial flow. The two non-cardiac cycle and prolonged compression cycle devices are reported to enhance arterial flow in part by reducing tissue fluid and/or venous content and thus venous resistance, to improve arterial inflow.

Another patent application US 2012/0065561 A1, titled as “device, system, and method for the treatment, prevention and diagnosis of chronic venous insufficiency, deep vein thrombosis, lymphedema and other circulatory conditions” relate to a compression device for applying compression to an extremity of a mammal includes a cuff adapted to be placed around and secured to the extremity. A control and tensioning unit is attached to the cuff and is operable to control a tension of the cuff to thereby control the compression applied to the extremity. The cuff may further include a bladder system, in which case the compression device further includes a hydraulic pump that is operable to transfer fluid within the bladder system to control the compression applied to the extremity.

Another patent application US 2007/0167884 A1, titled as “Method and apparatus for negative pressure therapy”, relates to an edema and/or wound treatment device, comprising a flexible material capable of forming an air seal circumferentially around a region of the body; a vacuum source coupled to said flexible material for creating or maintaining negative pressure within said sealed region; wherein said vacuum source aids in the reducing of edema, improving venous return, facilitating wound healing, and/or protecting skin.

Another US Patent Application published as US 2017/0312165 A1, titled as “adaptive compression therapy systems and methods”, relates to a system for providing compression treatment to a subject, the system comprising: a wearable compression device configured to provide compression to a body part of the subject, the wearable compression device comprising a compression plate, one or more motors disposed on the compression plate, one or more compression mechanisms configured to be wrapped around the body part and tightened or loosened by the one or more motors, one or more sensors configured to measure physiological data and/or device performance data, a controller for controlling the one or more motors according to a set of parameters, and a wireless communications module in communication with the controller and the one or more sensors; and a remote device configured to wirelessly communicate with the wireless communications module of the wearable compression device and to receive the measured physiological data and/or device performance data and to modulate the set of parameters for controlling the one or more motors.

One U.S. Pat. No. 925,665B2 relates to inflatable off-loading wound dressing assemblies, systems, and methods. To offload forces from a tissue site being treated with reduced pressure, an offloading pressure component may be used as part of a reduced-pressure treatment system. A reduced-pressure source of the reduced-pressure treatment system vents positive pressure exhaust into the offloading pressure component to inflate the offloading pressure component. The offloading pressure component disperses forces away from the tissue site. Other devices, methods, and systems are presented.

One other patent application US20180021178A1, titled as “Reduced-Pressure Systems, Dressings, Pump Assemblies and Methods” relates to a reduced-pressure wound treatment system includes a dressing and a pump assembly. An illustrative embodiment includes a reduced-wound pressure treatment system including a dressing with a rigid outlet coupled to a manifold, and a pump assembly including a pump and a rigid inlet, the inlet of the pump assembly being removably connectable to the outlet of the dressing by rigid interlocking formations. Another illustrative embodiment of a reduced-pressure wound treatment system includes a dressing with an outlet, and a pump assembly with an inlet, where the dressing is removably coupled to the pump assembly solely by the inlet being attached to the outlet, and the dressing cover and pump cover contact each other while the inlet and the outlet are attached together. According to a further illustrative embodiment, a reduced-pressure wound treatment system employs an inductive charger operably electrically charging a pump battery.

None of the aforementioned devices has shown ongoing established reliable ability to improve circulation and salvage ischemic limbs or promote rapid healing of wounds in studies. They are, as of the time of filing hereof, considered to be investigational by most health insurance systems.

None of the prior art designs have capability to produce a sequential, proximal to distal waveform that mimics and enhance normal physiologic flow and applies negative or variable pressure to wounds and to tissue adjacent to wounds to help facilitate healing.

Unlike the above-mentioned prior arts, the present invention provides an apparatus and method for pulse cycle pressure modulation and negative pressure therapy, wherein a negative pressure source, generated by the air compression system, provides sub atmospheric effect, that is applied to the distal limb during compression treatments. The negative pressure is mediated via a flexible chamber that surrounds the foot. The negative pressure acts to further enhance antegrade, i.e., distal limb blood flow. It accomplishes this by further increasing the pressure gradient along the long axis of the limb. The negative pressure is varied in a cardio synchronous manner. This is a unique feature, not found in other limb compression systems. Various forms of pressure techniques are available but using a combination of both positive pulsatile pressure along the limb's long axis to push blood downstream and application of negative pressure at the distal limb to further enhance flow delivery is novel and non-obvious.

The present invention provides an apparatus for pulse cycle pressure modulation and negative pressure therapy in a cardio synchronous manner to improve distal limb blood flow.

The present invention provides a method for pulse cycle pressure modulation and negative pressure therapy in a cardio synchronous manner to improve distal limb blood flow.

The present invention provides an apparatus for pulse cycle pressure modulation and negative pressure therapy comprising means for exerting negative pressure at the distal segment of the organism's limb, e.g., the foot or hand; wherein the negative pressure is varied in time relation to inflation of the immediately proximal limb pneumatic compartment.

The present invention provides an optionally modified apparatus for pulse cycle pressure modulation and negative pressure therapy comprising means for exerting negative pressure at the tissue of such body segment, suffering from incisional wounds or bedsores.

The present invention provides an optionally modified apparatus for pulse cycle pressure modulation and negative pressure therapy comprising means for exerting negative pressure at the tissue of such body segment, suffering from nonhealing vascular ulcer, sickle cell ulcers or diabetic ulcer.

The present invention provides an optionally modified apparatus for pulse cycle pressure modulation for organ transport and keeping organs perfused. The current perfusion systems only focus on infusing pulsatile fluid and not giving external synchronized pressure modulation to push venous and interstitial fluid out of the harvested organ. The same principles apply for optimizing microcirculation of artificial organs or organ perfusion outside the body.

The present invention provides an apparatus connected to a conduit supplying negative pressure, stored in a negative pressure reservoir and consequently provides said negative pressure to the target limb region via a valve, provisioned for opening and closing as designed by the device's default system or algorithm.

The present invention provides an apparatus for pulse cycle pressure modulation and negative pressure therapy; wherein negative pressure in the most distal limb pneumatic compartment is activated to enter that cell when positive pressure is changed to the immediately adjacent proximal compartment.

The present invention provides an apparatus for pulse cycle pressure modulation and negative pressure therapy; wherein negative pressure in the most distal limb pneumatic compartment is activated to enter that compartment at a time in continuous sequence with the sequential inflation of more proximal pneumatic compartments.

The present invention provides an apparatus for pulse cycle pressure modulation and negative pressure therapy, wherein the negative pressure is adapted to apply progressively decreasing negative pressure to the limb.

The present invention provides an apparatus for pulse cycle pressure modulation and negative pressure therapy; wherein the means for applying the negative pressure to at least one vertebrate organism's limb comprises the multi-compartment pneumatic device, having fluid conduits between adjacent compartments.

The present invention provides an apparatus for pulse cycle pressure modulation and negative pressure therapy; wherein the fluid conduits are sized to provide progressive changes in negative pressure within each more distal compartment when such pressure is applied to the adjacent proximal compartment.

The present invention provides an apparatus for pulse cycle pressure modulation and negative pressure therapy; wherein each fluid conduit comprises a valve.

The present invention provides an apparatus for pulse cycle pressure modulation and negative pressure therapy; wherein each valve changes state in response to a threshold pressure in the adjacent proximal compartment.

The present invention provides an apparatus for pulse cycle pressure modulation and negative pressure therapy; wherein each valve has an electrically controllable negative pressure threshold.

The present invention provides an apparatus for pulse cycle pressure modulation and negative pressure therapy; wherein a separately controlled source of negative pneumatic pressure is provided to each pneumatic device.

The present invention provides an apparatus for pulse cycle pressure modulation and negative pressure therapy; wherein the means for applying negative pressure to the limb comprises a plurality of hollow soft walled cells that are applied circumferentially around the limb, longitudinally adjacent to each other, along the long axis of the limb, and wherein said hollow soft walled cells are also connected via a fluid conduit to a regulated source of fluid under negative pressure, as well as having connectivity via a fluid conduit to a means to neutralize cell fluid contents with ambient pressure in a regulated manner.

The present invention provides an apparatus for pulse cycle pressure modulation and negative pressure therapy; wherein the means for applying negative pressure to at least one vertebrate organism's limb is operable to be applied to the limb in a time interval beginning upon release of negative pressure from the limb's most distal pneumatic compartment.

The present invention provides an apparatus for pulse cycle pressure modulation and negative pressure therapy; wherein the means for initiation of application of negative pressure to the limb's near distal pneumatic compartment begins upon receipt of a signal upon release of application of negative pressure in the limb's most distal pneumatic compartment.

The present invention provides an apparatus for pulse cycle pressure modulation and negative pressure therapy; wherein the received signal is a detected signal indicative of the device's signal to relieve negative pressure in the limb's most distal compartment.

The present invention provides an apparatus for pulse cycle pressure modulation and negative pressure therapy; wherein the predetermined time interval for application of negative pressure to each successive pneumatic compartment, going from near distal to most proximal, is determined by the default value or algorithm to be applied so as to be completed immediately before the next proximal to distal pneumatic compression cycle begins.

The present invention provides an apparatus for pulse cycle pressure modulation and negative pressure therapy; further comprising means for determining the amount of negative pressure applied to the limb.

The present invention provides an apparatus for pulse cycle pressure modulation and negative pressure therapy; further comprising a means to control the rate of negative pressure application and release.

The present invention provides an apparatus for pulse cycle pressure modulation and negative pressure therapy; further comprising a means to control the rate or velocity at which the onset of inflation of each successive pneumatic compartment after its most proximal compartment inflation begins.

The present invention provides an apparatus for pulse cycle pressure modulation and negative pressure therapy; wherein it is provisioned for identifying the PPG triggering signals, corresponding to which real time initiation of inflation of the most proximal air cell is timed towards optimizing the effect of said pressure therapy.

The present invention provides an apparatus for pulse cycle pressure modulation and negative pressure therapy; wherein it is provisioned for creating and optimizing the PPG waveform in real time, corresponding to the blood flow sensed by the sensor.

### SUMMARY OF THE INVENTION

The present disclosure of the invention relates to an apparatus and method for pulse cycle pressure modulation and negative pressure therapy in a cardio synchronous manner to improve distal limb blood flow; wherein the system is provisioned for applying negative or variable pressure to a portion of the body. The invention particularly relates to methods and apparatus for the application of a sequential and gradient pulse wave for treating wounds that are difficult to heal or livelihood limiting claudication and/or ischemic rest pain. An integral part of the present invention includes methods to determine blood flow and effects of treatments on tissue.

The system of the present invention is also provisioned to apply negative pressure therapy of distal wounds, particularly in combination with application of positive cuff pressure.

A negative pressure source, generated by the air compression system, provides this subatmospheric effect, that is applied to the distal limb during compression treatments. The negative pressure is mediated via a flexible chamber that surrounds the foot. The negative pressure acts to further enhance antegrade, i.e., distal limb blood flow. It accomplishes this by further increasing the pressure gradient along the long axis of the limb. The negative pressure is varied in a cardio synchronous manner. This is a unique feature, not found in other limb compression systems.

The apparatus of instant invention provides several novel innovations to help improve the ischemic limb that are non-obvious. Tissue viability is maintained in healthy individuals in part via the combined activities of cardiac pumping and the supply system of the circulation, arteries and capillary beds. In patients with diseased circulation, several components of the normal circulation may be impaired. These problems may include obstruction of named arteries and thickened capillary basement membranes, just to name two examples. The device of present invention addresses all such complications and is capable of facilitating effective flow of arterial blood, venous blood-flow, normalized fluid pressure in lymphatic system and consequently assisting the faster treatment of the wound or edema.

The apparatus of instant invention provides sequential pneumatic compression of the limb to enhance impaired antegrade flow. As described in the Starling-Landis theory of circulation, the arterial side head of pressure provides the hemodynamic energy that pushes arterial blood into capillaries to feed oxygen and nutrients to tissues. In the presence of named artery obstruction, as, for example, in atherosclerosis, that head of pressure is diminished. The apparatus of instant invention compensates for that decrease by adding extra pressure to push blood into capillaries.

The apparatus of instant invention uniquely addresses impaired blood flow in patients, particularly by the application of negative pressure at the tissues of the distal end of the limb towards addressing the venous and lymphatic fluid return issues. It also improves overall blood flow circulation and enhances wound healing. Using a newly developed pedal sleeve that encases the foot, the device of the present invention provides negative pressure to the foot during treatments. Negative pressure has the de facto effect of lowering distal tissue pressure. That lower pressure enhances the circulation by helping to draw blood flow into the distal limb and wound, thus further improving the circulation.

Negative pressure has the effect of lowering venous side pressure, to further enable capillary blood flow through tissues. Negative pressure thus helps circumvent pathologically elevated central venous pressure to improve the ‘return side’ component of the circulation.

The preferred embodiment of the present invention towards pulse cycle pressure modulation and negative pressure therapy comprises an apparatus for creating negative pressure directly at the tissue of the distal end of the limb, particularly in a cardio synchronous manner to improve distal limb blood flow. The apparatus comprises of a double walled stretchable/expandable chamber to be mounted around the end part of the limb, namely foot or hand; wherein said chamber is connected to a suction pump through at least one conduit and related peripheral components, namely fluid-pressure reservoir(s), air/fluid pressure monitor within the said chamber and its regulator-mechanism. The air/fluid pressure within the said double walled stretchable/expandable chamber is effectively monitored and regulated through a microcontroller-based controller module, which actuates and stops the action of air/fluid suction pump in accordance with the cardiological blood-flow signal inputs, mainly provisioned to be recorded in the form of PPG input signals and/or Doppler input signals and/or optionally in the form of EKG input-signals. The preferred embodiment of the apparatus for negative pressure therapy is provisioned to vary the air/fluid pressure within the double walled stretchable/expandable chamber and subsequently within the tissue enclosed within the chamber vary rapidly, at the same rate of heart-beat of the patient.

In one preferred embodiment of the present invention towards pulse cycle pressure modulation and negative pressure therapy comprising means for exerting negative pressure at the distal segment of the organism's limb, e.g., the foot or hand; wherein the negative pressure is varied in time relation to inflation of the immediately proximal limb pneumatic compartment. The apparatus connected to a conduit supplying negative pressure, stored in a negative pressure reservoir and consequently provides said negative pressure to the target limb region via a valve, provisioned for opening and closing as designed by the device's default system or algorithm. The negative pressure in the most distal limb pneumatic compartment is activated to enter that cell when positive pressure is applied to the immediately adjacent proximal compartment. The negative pressure in the most distal limb pneumatic compartment is activated to enter that compartment at a time in continuous sequence with the sequential inflation of more proximal pneumatic compartments.

The preferred embodiment of the apparatus for negative pressure therapy is so provisioned that the negative pressure is adapted to apply progressively decreasing negative pressure to the limb. The means for applying the negative pressure to at least one vertebrate organism's limb comprises the multi-compartment pneumatic device, having fluid conduits between adjacent compartments. Each of the fluid conduits comprises a valve; wherein each valve changes state in response to a threshold pressure in the adjacent proximal compartment. Each of valves, connected to fluid conduits of the negative pressure therapy apparatus has an electrically controllable negative pressure threshold; wherein a separately controlled source of negative pneumatic pressure is provided to each pneumatic device involved therein.

In one embodiment of the apparatus for negative pressure therapy, means for applying negative pressure to the limb comprises a plurality of hollow soft walled cells that are applied circumferentially around the limb, longitudinally adjacent to each other, along the long axis of the limb, and wherein the hollow soft walled cells are also connected via a fluid conduit to a regulated source of fluid under negative pressure, as well as having connectivity via a fluid conduit to a device to neutralize cell fluid contents with ambient pressure in a regulated manner.

The device for applying negative pressure to at least one vertebrate organism's limb is operable to be applied to the limb in a time interval beginning upon release of negative pressure from the limb's most distal pneumatic compartment.

In one embodiment of the apparatus for negative pressure therapy, the means for initiation of application of negative pressure to the limb's near distal pneumatic compartment begins upon receipt of a signal upon release of application of negative pressure in the limb's most distal pneumatic compartment. The received signal is a detected signal indicative of the device's signal to relieve negative pressure in the limb's most distal compartment. The predetermined time interval for application of negative pressure to each successive pneumatic compartment, going from near distal to most proximal, is determined by the default value or algorithm to be applied so as to be completed immediately before the next proximal to distal pneumatic compression cycle begins.

In one embodiment of the apparatus for negative pressure therapy, the apparatus for pulse cycle pressure modulation and negative pressure therapy further comprises apparatus for determining the amount of negative pressure applied to the limb. The apparatus further comprises apparatus to control the rate of negative pressure application and release.

In one method of treating a patient through pulse cycle pressure modulation and negative pressure therapy, wherein providing a two-pronged approach to treating poor circulation, that is, at one side enhancing arterial supply and improving venous return, the functioning of the system of present invention provides novel mechanisms that result in faster wound healing and limb salvage.

Negative-pressure wound therapy is generally considered to be a therapeutic technique that use a vacuum dressing to promote healing in acute or chronic wounds and enhance healing of first and second-degree burns. The therapy involves the controlled application of sub-atmospheric pressure to the local wound environment, using a sealed wound dressing connected to a vacuum pump.

Per conventional understanding of negative pressure therapy treatment, the continued vacuum draws out fluid from the wound and is supposed to increase the blood flow to the area. The negative pressure also prevents the return of the blood from the surface back to the body. Thus, after few cardiac cycles, the area gets engorged with blood and thus blocks the flow of fresh blood to the area. Once the area is engorged the continued suction pressure causes the seeping out of body fluids from the damaged skin surface or the wound. Seepage of body fluids cause additional problems. Accumulation of excessive and stagnant fluid in the wound predisposes to increased microbial growth and added risk of infection. Thus, use of negative pressure requires frequent dressing changes that itself increases the risk of infection. To counteract this risk an antibiotic dressing or sterile environment is needed. The vacuum can also be applied intermittently, for a fixed period of time without taking blood flow patterns into consideration. Depending on when suction pressure is started in relation to the cardiac cycle they may enhance or impede blood flow and or increase the seepage of body fluids from the wound.

Therefore, the method of present invention involving the apparatus of the present invention enhances the blood flow through the area while preventing the retention of blood, which is an inherent flaw of continuous or non-cardio-synchronized suction devices. The discontinuation of negative pressure during the diastolic phase of cardiac cycle will allow the blood to flow back towards the heart thus making room for new blood bringing oxygen and fresh nutrients. In addition, the relaxation phase of our pulsatile blood flow augmentation system enables drainage of venous blood, will prevent engorgement of tissues and thus prevents seepage of body fluids from the damaged skin surface. This will help keep the wound relatively dry and prevent the inherent complications of excessively wet wounds while enhancing the blood flow to and through the wound surface. Likewise, non-healing surgical wounds like sternotomy and laparotomy wounds can be addressed.

It is possible that the pressure therapy device of present invention can optionally be applied to any other body parts outside of the leg including arms to improve blood circulation at the distal ends, to treat, e.g., Raynaud's, frostbite and so forth, and upon the torso to improve blood circulation at any areas with reduced blood flow, as well as, conceivably, the carotid arteries if compression of trachea can be avoided, so as to treat hair regrowth, eye disorders, and brain diseases and disorders including Alzheimer's which is being considered a form of microvascular dementia.

Ostomy surgery is performed to redirect the body's waste products due to disease, trauma or defects in the colon, bowel and bladder. Ostomy wounds are difficult to heal in part because of edema, low blood flow and recurrent infection due to the wet tissue impede the healing of the wound. The pressure therapy device of present invention can optionally be applied to ostomy wound to decrease edema and enhance blood flow, thus healing the ostomy wound.

Lipedema is a disorder where there is enlargement of both legs due to deposits of fat under the skin. In severe cases the trunk and upper body may be involved. Various causes have been proposed including microcirculatory disturbances and abnormal lymphatic flow. The pressure therapy device of present invention can optionally be applied to the affected area to enhance lymphatic flow and correct microcirculatory disturbances, thus providing relief to the patients.

Certain areas of the body have inherently low blood flow so that any trauma or surgery of those areas can lead to difficult to heal wounds. Sternum, breast, cornea, scarred tissue and surgery involving the intestines can result in a nonhealing wound. Patients with diabetes or a weakened immune system also face a higher risk of bacterial infection and non-healing wounds. Additionally, areas with post injury infection can lead to edema and/or scarring of adjoining wound tissue leading to poor blood flow to the wound. The continued inflammation, injury and edema makes these wounds difficult to heal even after the infection has been treated. The pressure therapy device of present invention can optionally be applied to the affected area to enhance blood flow to the tissue thus correcting the microcirculatory imbalance and promote healing.

Osteomyelitis or the infection of the bone is difficult to heal because of inflammation, edema and low blood flow. Given the low blood flow to the infected area, antibiotics cannot reach the infected tissue to treat the infection. Associated edema further compromises the microcirculation. In certain situations where the infection causing organism is known and responsive to antibiotics, the blood flow to the infected tissue can be enhanced using the pressure therapy device of present invention. Enhancing the blood flow to the infected area will lead to increased penetration of antibiotics, thus helping to treat osteomyelitis.

Stump ulcers are common problems in amputees. Temporary discontinuation of prosthetic limb use is frequently employed to facilitate healing. Post-surgical injury and recurrent trauma due to prosthesis use leads to inflammation and edema, making stump ulcers difficult to heal. The pressure therapy device of present invention can optionally be applied to the affected area to enhance blood flow to the tissue and promote healing.

It is being increasingly recognized that obesity and inflammation are interrelated with metabolic syndrome. As the fat deposit increases, the neovascularization or blood supply lags behind the expanding fat accumulation, leading to hypoxia of the fat deposits. Worsening of hypoxia lead to inflammation and chronic inflammation is thought to lead to the metabolic syndrome. Inflammation leads to edema which compromised blood flow and worsens microcirculatory changes and lymphatic flow, thus further worsening the inflammation of the adipose tissue. The pressure therapy device of present invention can be applied to the affected obese area to enhance blood flow to the adipose tissue and decrease edema thus correcting the microcirculatory imbalance and inflammation, and possibly correct abnormalities associated with the metabolic syndrome. Resulting decrease in edema and inflammation, can be used in theory to treat difficult to remove fat deposits in the body.

Degenerative joint disease is multifactorial in etiology. However, it is being increasingly recognized that microcirculatory changes and edema have a significant causative role. It has been noted that early subchondral changes include redistribution of blood supply with marrow hypertension, edema and probably micro-necrosis, factors that likely contribute to or aggravate degenerative joint disease. The pressure therapy device of present invention can optionally be applied proximal to the affected joint area to enhance blood flow to the joint and cartilage and decrease edema, thus correcting the microcirculatory imbalance and promote healing.

Mesenteric Lymphatic flow and edema has been implicated in the pathogenesis of the inflammatory bowel disease. Alterations in the intestinal lymphatic flow is a well-established feature of human inflammatory bowel disease. The pressure therapy device of present invention can optionally be applied to the abdomen of the affected patients to enhance Lymphatic drainage of the mesentery and correct the microcirculatory imbalance and promote healing of the inflammatory bowel disease.

One optionally selected embodiment of the said pressure therapy device of the present invention can be applied further to one optionally selected area of the body to treat, heal, or affect other areas of the body. Blood flow improvement in one area can have a parallel affect in the contralateral limb and/or allow better overall blood flow to the entirety of the body.

Therefore, the pressure therapy device of the present invention can optionally be employed in selective manner towards treating impaired blood flow and regulating lymphatic flow in patient's different body parts, leading to healing in patients in a myriad of conditions, particularly in at least one of the following conditions:
1. Hair growth on scalp
2. Increased brain blood flow in age related memory disorders
3. Stroke: selectively compressing carotid artery and preventing pressure on the trachea so the person can continue to breathe
4. Visual loss due to vascular diseases of eye
5. Sinus blood flow to heal sinus infections
6. Carotid sinus pressure or massage can be used to treat hypertension and increase parasympathetic tone/discharge in the body

### BRIEF DESCRIPTION OF THE DRAWINGS

FIG. 1: Represents complete schematic plan for applying the components of the system for pulse cycle pressure modulation and negative pressure therapy; particularly towards enhancing arterial blood-flow and also for improving venous return;

FIG. 2: Schematic presentation of the inflatable double walled plastic chamber (foot model), showing its structural and functional provisions for negative pressure therapy, directly employed at the distal tissues of the limb;

FIG. 3: Schematic graphical representation of arterial and venous blood flow and their respective enhancements, demonstrating an illustrative scenario of treating a patient with impaired blood flow and suffering from edema of distal wound; particularly wherein various waveform inputs may comparatively be analysed towards understanding the effect of applying the positive and negative pressure therapy during the cardiac cycle using the apparatus of the present invention, involving Arterial Pulse Enhancement Technology (APET) and Venous Flow Enhancement Technology (VFET) principals of pressure therapy of the present invention as explained hereinbelow.

### DETAILED DESCRIPTION OF THE EMBODIMENT OF THE INVENTION

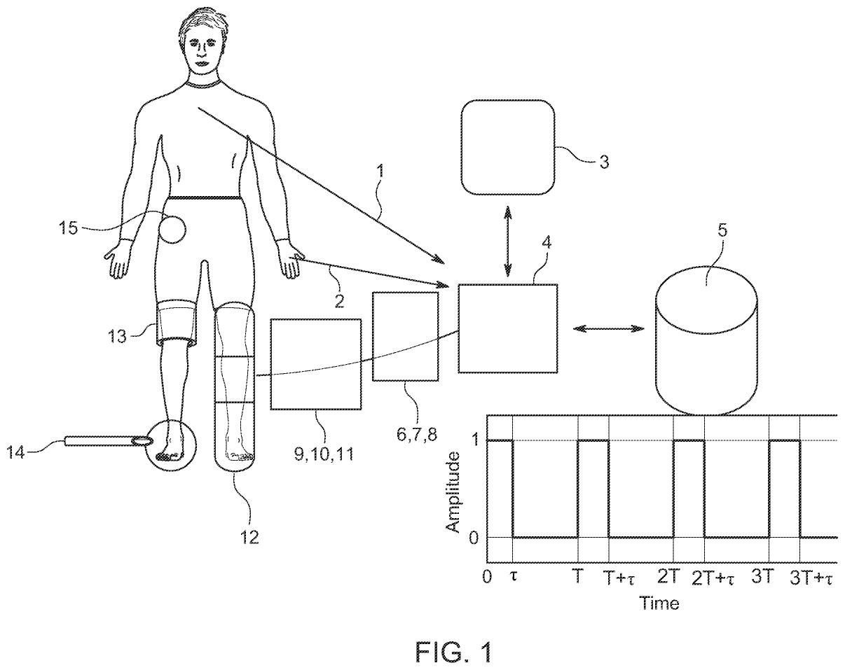

Referring to FIG. 1 in more detail, Simplified block diagram of device. Below is identification of all the components in FIG. 1.

1. EKG signal

2. Blood flow sensor, actuating PPG signal,

3. User interface

4. Computing system with the controller module for the system

5. External database

6. Air Compressor

7. Air Reservoir

8. Inflation valves

9. Conduits

10. Circuitry controlling inflation valves

11. Conduits

12. Infrared sensor acquires input of tissue infrared emission.

13. Inflation Air Cells (in Compression Cuff)

14. Inflatable double walled plastic chamber at foot

15. Optionally selected blood flow sensor at the proximal end of the limb

16. inner shells of the Inflatable double walled plastic chamber

17. outer shell of said double walled chamber (14)

18. the valve (18) for inflation of said double walled chamber (14)

19. Pressure modulation port of the double walled chamber (14)

20. Optional blood flow sensor (preferably for PPG signal), to be mounted selectively at the wound tissue region, where negative pressure is applied

21. represents distal end of the limb or hand (or other optionally selected body-part), where the negative pressure is applied

22. closable component of the chamber (14), provisioned for mounting and holding the limb for negative pressure therapy

23. That region of the closable component (22) where inner shell (16) and outer shell (17) are sealed/integrated together.

FIG. 1 depicts a simplified overall design of the sequential compression system, including the apparatus for negative pressure therapy. The system compresses a limb at specific time intervals during the cardiac cycle as determined by inputs to the system. By blood flow sensors, namely sensor (1) for actuating EKG signal (1) or sensor (2) for actuating PPG signal, inputs to the system are selected from at least in one form, namely the EKG or photo plethysmograph (PPG) waveform. EKG or PPG leads are attached to the body to supply the heart's pulsatile signal. At the distal end of the limb where the blood flow increase is desired, a PPG monitor [not seen in the figure} is attached to detect pulsatile blood flow in the underlying tissue. Based on the cardiac blood-flow response in the form of PPG waveform inputs, at the distal limb the computing system with the controller module for the system (4), supported with the provisioned database (5) therein activates and regulates the inflatable components to provide optimal pulse cycle pressure modulation and negative pressure therapy. A series of inflatable air cells (13) are wrapped around the limb. Inflation of air cells occurs from fluid filling the cell through fluid traversing from the Air Reservoir (7) through Conduits (9) through the Inflation Valves (8) through Conduits (11) into the Air Cells (13); Fluid releases from the Air Cells (or compression cuff) (13) through conduits to Deflation Valves (not depicted). Fluid is generated by an air compressor (10) to maintain fluid under high pressure in the Reservoir (7).

One important component of the present system for pulse cycle pressure modulation and negative pressure therapy is at least one sensor (1, or 2 or 15 or 20), which provides feedback necessary to deliver higher quality treatment. Said blood flow sensor may be selected from at least one of the following factors:
a. Vibrations: a vibration sensor can be used to sense the start of the movement of the blood. This may not be the actual blood flow.
b. Indwelling pressure sensor catheter: there are catheters which can be put into major vascular structures close to the site of treatment which will sense the actual pressure and the feedback from those pressure can help to guide the adjustment in the cuff pressure.
c. Wave form at selected point: the heart pumps the blood in the classic waveform, the waveform as Normal Pulses, is critical to mimic physiological functioning. Various points on the waveform if enhanced may have different effect on the ultimate microcirculation of the treatment part. A sensor programmed pickup of the waveform and start compressions based on the waveform or the compressions based on the position of the waveform as predicted by the structure of the previous waveform. Once the change in the pressure has been applied, the waveform changes in a predictable manner. This can be used to further modify and fine-tune the end waveform will have the physiological effect for an individual patient.
d. EKG signal: the electrical activity of the heart travels faster than the actual blood and with current sensors can be picked up from different parts of the body including the extremities, chest or a combination of multiple points.
e. Doppler flow: can detect the actual flow of blood under the sensor and alteration of the actual flow by the changes in the pressure can be used to further modify the pressure to a desired goal.

Smart materials are designed materials that have one or more properties that can be significantly changed in a controlled fashion by external stimuli, such as stress, temperature, moisture, pH, electric or magnetic fields. As the field of smart materials develops, materials are available which can stiffen with the passage of electric current. As the electric current can be passed very rapidly with the current electronic circuitry the speed and the options to mimic the pulse wave accurately is significantly enhanced. The use of such material leads to significant approximation to the natural state of pulse. This effect can be used to enhance the treatment potential of the invention.

Tourniquet using the invention to provide compression of the limb between the venous pressure and arterial pressure will engorge the veins without blocking/diminishing the arterial flow. The venous engorgement can be used for either placement of vascular cannulas or for blood draws. The venous engorgement applied over a long time can be used for enhancing the vein for fistula formation for dialysis.

There is a significant problem of syncope whenever high gravitational forces are applied to the human body. The blood starts pooling in the dependent portion of the body causing severe circulatory irregularities and can lead to stroke or syncope. This device can be connected to the thighs and arms to prevent excessive or diminished flow to the center of body in case of significant gravitational forces.

The Inflation System is comprised of the individual components: Air Compressor (6), Air Reservoir (7), Inflation valves (8), Conduits (9), Circuitry controlling inflation valves (10), Conduits (11), Inflation Air Cells (13), and can be replaced with other mechanisms that actuate compression onto the limb in a sequential manner. These include:
1) Nanotechnology of material which stiffens with electrical current or fluid or any other activator where the material would surround the limb to be compressed, have appropriate conduits to an actuator, and have the capability to compress at local areas along the entirety of the limb or area to be compressed.
2) Using the Inflation System of any fluid other than air that can compress the limb.

An alternative embodiment may also combine a sub-atmospheric pressure reservoir and vacuum pump, together with suitable valves and valve actuators with the Inflation System elements already described. By combining these elements, extremely rapid inflation-deflation cycles may be achieved by venting the inflation air cells to the low-pressure reservoir, rather than to the ambient atmosphere. Additionally, the sub-atmospheric pressure reservoir may be used in combination with vacuum wound healing dressings to provide wound treatment that overcomes the deficiencies inherent in prior art vacuum wound dressing systems.

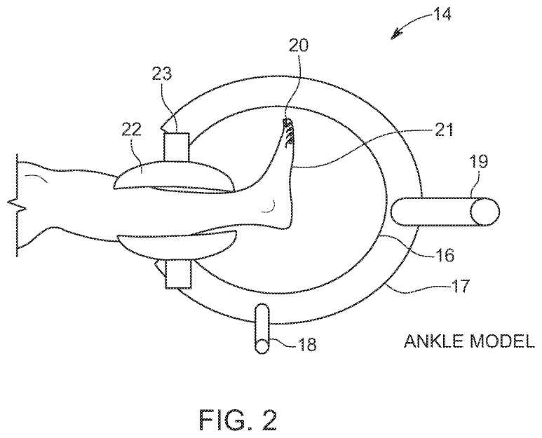

One of the preferred embodiment of the present system of the invention, as indicated in FIG. 1 includes the apparatus responsible for creating negative pressure, particularly directly at the tissues of distal limb portion, leading towards increased arterial blood flow and improved venous return. FIGS. 2 duly illustrate the said inflatable double walled chamber (14) for actuating negative pressure therapy. The most important component for negative pressure therapy is the inflatable double walled plastic chamber (14), to be mounted at the distal limp section, namely at the foot or below knee or around palm/below elbow etc. of the patient. Preferably said double walled chamber (14) comprises of two mutually stretchable or inflatable shells (16) and (17), both made of polymeric sheets. Once air or another suitably selected fluid is filled between two inflatable shells, inner shells (16) and outer shell (17) of said double walled chamber (14), using the valve (18) for inflation, it provides stiffness for outer shell (17) and will not allow collapse or bulging of said chamber (14) during treatment using positive or negative pressure.

In one of the embodiments of the present system of the invention, firstly the end portion of the patient's limb, usually leg or hand (21), is preferably mounted with a blood flow sensor (20) at the distal end of the limb placed within the double walled inflatable chamber (14). The distal end of said limb or hand (21) is inserted within the cavity of said double walled chamber (14) through the closable component (22) of said chamber (14), wherein said component (22) is provisioned for mounting and holding the limb for negative pressure therapy. Said closable component (22) of the chamber (14) is relatively rigid in comparison to the walls (16) and (17), wherein the inner surface of said component (22), which comes in direct contact with the skin of the limb or hand or any other region of body-part, comprises of a layer or lining of silicon sealant. Said silicon sealant or any other optionally selected material therein, provided with the surface of said closable component (22) is actually capable of creating a vacuum-based sealing with skin of the limb/hand or body-part of the patient, which is very much comfortable to the patient. The closable component (22) of chamber (14) actually integrates the inner shell (16) and outer shell (17) at its edges (23) and therefore it is comparatively more firm in structure. Said chamber opening end (22) of chamber (14) is so constructed that it comprises of padding of silicon sealant (23) for surrounding and sealing the limb (21), wherein said padding of silicon sealing is capable of preventing air leakage at the interface of limb (21) and the chamber opening end (22) during negative or positive pressure change within the inflated said chamber (14). Once the said chamber (14) is effectively mounted at distal region of the patient's limb and duly inflated, it is ready to respond to the controller modules (4) in real time to the signal inputs of patient's cardiac cycle, as recorded by the computing system therein and its database (5). Based on the real time signal inputs of patient's cardiac cycle, the air or fluid pressure is decreased or released through the pressure modulation port (19), directly connected to the suction pump used conduit.

It is pertinent to mention herein that the construction and dimension of said inflatable chamber (14) of the present system of the invention may optionally comprise of single wall and wherein said wall of the inflatable chamber (14) may support the creation of negative pressure around the limb portion (21), enclosed within the cavity of the said inflatable chamber (14). Principle behind selection of the shape, dimension and construction of the said chamber (14), particularly for a person skilled in the art, is that the inner cavity of said chamber (14) should effectively support air or fluid pressure variation therein and therefore creation of negative pressure directly on the tissues of distal region of the limb, enclosed within the said cavity, mainly in real time response to the signal inputs recorded for real time signal inputs of patient's cardiac cycle.

In view of the preferred embodiment of the system for negative pressure therapy of the present invention, it is important to analyse the impaired blood circulation system in a patient with edema or wound on his limb. Such analysis will help in understanding the present method of the invention involving the system for pulse cycle pressure modulation and negative pressure therapy.

It is a well-known fact that the circulatory system of a mammal includes the arterial blood flow system, venous return system, lymphatic system and capillary system. Any imbalance among the flow of blood, lymph or other fluid may be a reason for edema in a limb or impaired blood flow. It is the regular practice to apply cuff assisted compression devices for increasing the blood flow in the arterial blood flow system. However, due to said compression of limb the blood/fluid pressure within venous return system, lymphatic system and capillary systems may get adversely affected, which may lead to further irregularity in the circulatory system. As the blood starts flowing from the arterial system to the capillary system and further on to the venous system, it starts leaking fluid, which has to be reabsorbed back by the venous system.

The tissue pressure which forces the fluid from tissue into the blood vessel starts rising when there is injury to the tissue. Pressure applied from outside the limb to increase tissue pressure to decrease edema has been used in the form of pressure bandage. Since it applies pressure in both systolic and diastolic phase of the blood flow it decreases the swelling but it also decreases the microcirculation flow. Since it prevents flow of plasma from coming out of blood vessel during the systolic phase, it compromises the nutrients and oxygen delivery to the inflamed tissue leading to additional ischemic injury and thus delays healing.

Pressures within the capillary system varies along the course of the capillary from its arterial to its venous end, as blood flows through the capillary, where various influences impact on intracapillary fluid flow. As fluid flows through the capillary, its pulsatility gets progressively more blunted. By the time fluid reaches the distal part of the capillary, the venous side, pulsatility is significantly diminished. The entire system of capillary flow, into and out of the surrounding interstitium and soft tissues, creates a system which is critical to provide nutrition to those tissues. This system is described in the Starling-Landis theory of fluid flow through capillaries. The pressure within the capillary system also varies with time as the pulse wave advances. As the pulse wave passes through the capillary from the arterial side to the venous side, the fluid moves out of the arterial end during the peak of the arterial pressure and moves back into the capillary at its far end, the venous end, where the intracapillary pressure is lower.

Lymphatic flow decreases with externally applied continuous pressure as continuous pressure collapses the collecting system and does not allow the lymphatic system to fill. In contrast cardio synchronized externally applied limb pressure allows the lymphatic system to fill and thus enhance its flow. Similar principles apply to the venous system, although the one-way valves in the venous system are less and are only in larger vessels.

Synchronized compression will move the fluid sequentially through the lymphatic system as it has multiple connected chambers with one-way valves. Whereas continuous pressure does not allow lymphatics to fill, application of intermittent pressure to a limb will fill lymphatics repeatedly and the next cycle of pressure will push the collected fluid in the lymphatic chamber out to the next lymphatic chamber, thus enhancing lymphatic flow. The increased flow of fluid through the lymphatic system provides the much-needed nutrition and oxygen to maintain optimal health of the lymphatic system. This has a potential role in antiaging therapies.

Inflammation causes exuding of protein-rich fluid from the capillary micro circulation. As part of the inflammatory process leaking blood vessels enable fluid to move out of capillaries into the interstitium. By selectively enhancing pressure at the wound site, the instant device in consideration can help push interstitial fluid back into the microcirculation. This has the potential of reducing edema at the site of the injury.

Therefore, with regard to highlighting important advantages of the present invention, one exemplary case-analysis relating the limitations of the conventional treatment of Edema is described:

The edema of the wound is a very difficult problem to treat. There are several factors which determine the blood flow to the skin and various tissues. Some of the major such factors include:
a. Tissue pressure;
b. Tissue oncotic pressure;
c. Blood pressure;
d. Blood oncotic pressure;
e. Venous pressure;
f. Arterial pressure, and
g. Colloidal pressure.
h. Capillary wall permeability.

Solution using the instant invention:

Current treatments for edema utilize applying a bandage throughout the pulse cycle. The pressure is increased for both arterial and venous side or the trough and the peak of the pulse wave. The pressure counteracts the pulse pressure, causing decrease in the flow of interstitial fluid, which compromises the provision of nutrients to the tissue, with resultant poor perfusion and risk of infection and ulcer formation.

Cardio synchronized compression at the tissue site: If we selectively apply the pressure at the tissue site only in phase with the cardiac cycle, it will allow the interstitial fluid to be pushed back into capillaries when the limb is compressed. If we use cardio-synchronized compressions to increase the tissue pressure fluid will be pushed back into the venous system where it will be drained, and thus removed from the tissue. This will decrease the tissue pressure, leading to increase blood flow and interstitial fluid flow during the arterial phase, providing better nutrients and oxygenation to the tissue.

Working mechanism of APET therapy and VFET therapy in wound healing are as under:

As per the present invention in an exemplary scenario, the principle of pressure therapy towards treating an edema of the wound is as under:
Principle of pressure: positive pressure and negative pressure
APET Therapy: positive pressure at thigh and negative pressure at wound site, during systole; and
VFET Therapy: positive pressure at foot during diastole.
Indications for both pressures: Wound healing and Edema/inflammation treatment respectively

1. Arterial Pulse Enhancement Technology (APET): The arterial pulse can be enhanced in two ways:
a. Positive pressure proximal to the tissue to enhance the diastolic phase of the pulse pressure for the pulse wave passing beneath the tissue. e.g. Cuff on thigh for increasing blood flow to the leg. the classic APET technology. or PP-APET (positive pressure-arterial pulse enhancement technology); and

b. Negative pressure at the site of the tissue, during the systolic phase, to bring more blood to the tissue area. NP-APET or Negative pressure arterial pulse enhancement technology. This can be applied by placing a negative pressure device around the wound during the systolic phase of the pulse flow. This will increase the tissue perfusion and interstitial fluid flow during the systolic phase of the pulse.

2. Venous Flow Enhancement Technology (VFET): Venous flow can be enhanced by applying cardio synchronised positive pressure during the diastolic phase of the pulse. External Pressure which correlate with tissue pressure rises and pushes the fluid back inside the venous system. Tissue pressure enhancement during the diastolic phase moves the fluid from the interstitial tissue into the venous system, decreasing the edema. The VFET can be used for two indications:

a. To enhance the venous drainage

b. to enhance the lymphatic drainage

VFET should not be used in uncontrolled infected tissue due to risk of spreading the infection to blood or causing septicemia.

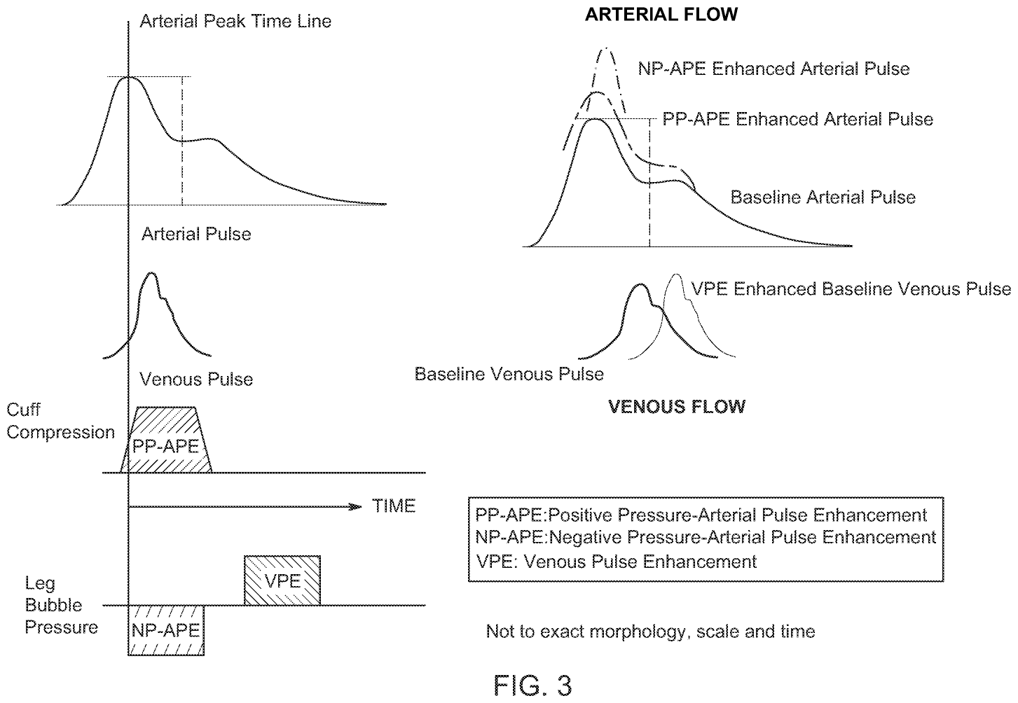

FIG. 3 explains an illustrative scenario of treating a patient with impaired blood flow and suffering from edema of distal wound; wherein various waveform inputs are comparatively analysed and accordingly the apparatus of present invention and its controller module is calibrated towards pulse cycle pressure modulation and negative pressure therapy, particularly to mimic and enhance the natural arterial blood flow or venous return towards healing the said impaired blood flow complications and/or the distal wound. EKG or PPG leads are attached to the body to supply the heart's pulsatile signal, the EKG or PPG. At the distal end of the limb where the blood flow increase is desired, a PPG monitor is attached to detect pulsatile blood flow in the underlying tissue. Based on the cardiac blood-flow response in the form of EKG waveform inputs or PPG waveform inputs, the computing system with the controller module for the system (4), supported with the provisioned database (5) therein activates and regulates the inflatable components towards pulse cycle pressure modulation and negative pressure therapy.

From the elaborate graphical representation of FIG. 3, the effect of applying the pressure therapy using the apparatus of present invention, particularly involving the APET and VFET principles of pressure therapy of the present invention as explained hereinabove, is comprehensively understood. By applying the positive pressure through compression-cuff (12) at the limb during systole, the arterial blood flow gets increased, which is indicated by the enhanced waveform signal (i.e. by PP-APE Arterial Enhanced Pulse). Said arterial blood flow further increases by applying negative pressure at the wound site during systole through the Inflatable double walled plastic chamber (14), preferably simultaneously with the said cuff-compression at (12), as shown by waveform signal of NP-APE Arterial Enhanced Pulse. When positive pressure is applied at tissue site of leg, the venous flow also gets enhanced, as indicated in comparative manner by both the venous waveform signals, namely Baseline Venous Flow and the VFET Enhanced Baseline Venous flow.

While the invention has been described in its preferred embodiments, it is to be understood that the words which have been used are words of description rather than of limitation and that changes may be made within the purview of the appended claims without departing from the true scope and spirit of the invention in its broader aspects. Rather, various modifications may be made in the details within the scope and range of equivalents of the claims and without departing from the spirit of the invention. The inventors further require that the scope accorded the claims be in accordance with the broadest possible construction available under the law as it exists on the date of filing hereof (and of the application from which this application obtains priority, if any) and that no narrowing of the scope of the appended claims be allowed due to subsequent changes in the law, as such a narrowing would constitute an ex post facto adjudication, and a taking without due process or just compensation.

1. An apparatus for pulse cycle pressure modulation and negative pressure therapy treatment of a limb of a mammal comprising a pneumatic system for exerting negative pressure upon a tissue of the limb of the mammal which pressure is synchronized with a transduced real time signal representative of the mammal's cardiac cycle.

2. The apparatus of claim 1 wherein the apparatus alternately applies negative pressure to improve arterial blood flow and positive pressure to increase venous blood flow and the apparatus comprises a transducer for signal input of the cardiac cycle, a microcontroller-based control module and an associated computing system, an air compressor, an air reservoir, one or more inflation valves, conduits, electronic circuitry for controlling the inflation valves, and one or more inflation air cells adapted to compress tissue.

3. The apparatus of claim 2, wherein the inflation air cells comprise inflatable double walled chambers for exerting negative pressure on a mammalian limb.

4. The apparatus of claim 3, wherein the double walled chambers each comprise inner and outer inflatable shells wherein once a fluid is introduced between the inner inflatable shell and outer inflatable shell using a valve on the outer shell, the double walled chamber resists deformation during subsequent inflation or suction.

5. The apparatus of claim 3, wherein the double walled inflatable chamber is positioned over the mammal's limb, and the limb is padded and sealed to the inner shell by a curable material.

6. The apparatus of claim 1, wherein the negative pressure is varied in timed relation to a transduced signal that is indicative of cardiovascular activity.

7. The apparatus of claim 6, wherein negative pressure is exerted at the distal segment of the mammal's limb.

8. The apparatus of claim 7, wherein the negative pressure is varied in timed relation to inflation of the immediately proximal inflatable chamber.

9. A method for pulse cycle pressure modulation and negative pressure therapy, wherein at least one of the following steps is performed simultaneously or sequentially:
(i) applying positive pressure at a limb during systole;
(ii) applying negative pressure at a wound site during systole;
(iii) applying positive pressure at a tissue site on the limb during diastole.

10. The apparatus of claim 1, wherein the negative pressure is applied by progressively decreasing negative pressure to the limb.

11. The apparatus of claim 10 comprising a multi-compartment pneumatic device having fluid conduits between adjacent compartments.

12. The apparatus of claim 11, wherein the fluid conduits are sized to provide progressive changes in negative pressure within each more distal compartment when such pressure is changed in the adjacent proximal compartment.

13. The apparatus of claim 12, wherein each fluid conduit comprises a valve.

14. The apparatus of claim 12 wherein the pressure, flow rate, flow volume, and timing of fluid flows is selectively controllable.

15. The apparatus of claim 14, wherein the pressure, flow rate, flow volume, and timing of fluid flows varied in timed relation to a transduced signal that is indicative of cardiovascular activity.

16. The apparatus of claim 1, wherein the transduce signal is derived from a pulse oximeter.

17. A method of treating a vertebrate organism using the apparatus of claim 1 wherein the following treatment parameters may be independently varied:
(a) delay in time from largest detected signal of cardiac activity at which time the most proximal compartment inflation begins;
(b) exerting negative pressure at the distal segment of the organism's limb, e.g., the foot or hand or any other optionally selected body-part;
(c) maximal pressure in one or more compartments;
(d) duration of compartment inflation;
(e) time after inflation of most proximal compartment when the next compartment inflation begins;
(f) pressure of each successive compartment, relative to its most immediate neighboring proximal compartment; and
(g) duration of treatment.

18. A method of treating a wound of a vertebrate organism comprising the steps of (a) detecting cardiovascular activity of the organism; (b) applying varying sub-atmospheric pressures to a wound site of the organism in timed relation with the detected cardiovascular activity.

19. A method of treating one or more of the following human conditions: peripheral arterial obstructive disease; microangiopathy; vasculitis; vasa vasorum disease; complex regional pain syndrome; frostbite; erythromelalgia; trauma induced limb wound; chronic limb wound; diabetic neuropathic wounds; trench foot; Raynaud's disease; vasa nervosum disease; peripheral neuropathy; and peripheral arterial diseases in which traditional or standard treatments are contraindicated or not advised; by the steps of (a) detecting cardiovascular activity of the human; (b) applying varying sub-atmospheric pressures to one or more somatic sites on the human in timed relation with the detected cardiovascular activity.
